(19) United States
(12) Patent Application Publication
(10) Pub. No.: US 2014/0271920 A1
(43) Pub. Date: Sep. 18, 2014

(54) SKIN OINTMENT FORMULATION

(71) Applicant: Diana Jean Stroebe, Auburn Hills, MI (US)

(72) Inventor: Diana Jean Stroebe, Auburn Hills, MI (US)

(21) Appl. No.: 13/830,917

(22) Filed: Mar. 14, 2013

(51) Int. Cl. A61K 36/889; A61K 33/30; A61K 35/02; A61K 31/575

(52) U.S. Cl. CPC A61K 36/889; A61K 31/575; A61K 33/30; A61K 35/02
USPC 424/641

(57) ABSTRACT

An ointment formulation for treating a skin of a mammal includes an occlusive and nourishing ingredient in an amount configured to reduce moisture evaporation from and seal the skin, and supply amino acids to the skin. The formulation also includes an anti-pruritic ingredient in an amount configured to reduce itching of the skin. Additionally, the formulation includes an antiseptic and anti-oxidant providing ingredient in an amount configured to protect the skin against infection. Furthermore, the formulation includes a moisturizing ingredient in an amount configured to soothe and reduce dryness of the skin. A method of generating an ointment formulation to treat a skin disorder is also disclosed.

### TECHNICAL FIELD

The present disclosure relates to a formulation for an ointment to be applied to a skin of a mammal.

### BACKGROUND

Ointments or balms are frequently used to protect skin of mammals, as well for treating certain dermatologic conditions, disorders, and diseases. Such conditions may include inflammation, redness, cracking, dryness, insect bites, allergic reactions, various types of dermatitis, eczema, etc.

Generally, such ointments are specifically formulated using active agents mixed into a base that acts as a carrier for the active agents to address the particular malady and promote healing. Additionally, such ointments may include supplementary agents intended to impart a pleasant scent or aroma to what may otherwise be a malodorous substance.

The application of the ointment is typically performed by topically administering or otherwise applying the ointment to an affected area on the skin, in order to soothe, soften, and/or condition the affected area. The subject ointment is generally administered at regular intervals until the offending condition is relieved or the danger of such a condition has been removed.

### SUMMARY

An ointment formulation for treating a skin of a mammal is disclosed. The ointment formulation includes an occlusive and nourishing ingredient in an amount configured to reduce moisture evaporation from and seal the skin and supply amino acids to the skin. The formulation also includes an anti-pruritic ingredient in an amount configured to reduce itching of the skin. Additionally, the formulation includes an antiseptic and anti-oxidant providing ingredient in an amount configured to protect the skin against infection. Furthermore, the formulation includes a moisturizing ingredient in an amount configured to soothe and reduce dryness of the skin.

The occlusive and nourishing ingredient may be Lanolin, the anti-pruritic ingredient may be Zinc Oxide, the antiseptic and anti-oxidant providing ingredient may be coconut oil, and the moisturizing ingredient may be petroleum jelly. The coconut oil may be an organic substance.

The Lanolin ingredient may be provided in the range of 74-85% by volume. The Zinc Oxide ingredient may be provided in the range of 4-12% by volume. The coconut oil ingredient may be provided in the range of 5-9% by volume. The petroleum jelly ingredient may be provided in the range of 3-7% by volume.

Furthermore, the Lanolin ingredient may be provided at approximately 82% by volume. The Zinc Oxide ingredient may be approximately 5% by volume. The coconut oil ingredient may be provided at approximately 6% by volume. The petroleum jelly ingredient may be provided at approximately 6% by volume.

The ointment formulation may additionally include a plurality of essential oils collectively in a range of up to 2% by volume. Additionally, the plurality of essential oils may collectively be configured to promote cell preservation and tissue regeneration, provide an antiseptic function, relieve topical discomfort, inflammation, and itching, and impart a physically impacting and pleasing scent.

Furthermore, each of the plurality of essential oils may be in a range up to an approximate maximum of 0.8% of the formulation by volume.

The plurality of essential oils may include a Lavender essential oil in an amount configured to have an anti-inflammatory and anti-pruritic effect on the skin, reduce itching, and impart the physically impacting and pleasing scent to the formulation. The plurality of essential oils may also include a Helichrysum essential oil in an amount configured to promote skin regeneration and have a healing effect on the skin. The plurality of essential oils may additionally include a Cedarwood Atlas essential oil in an amount configured to have a seborrhea reducing and antiseptic effect on the skin. Furthermore, the plurality of essential oils may include a Frankincense essential oil in an amount configured to have a vulnerary effect on the skin by minimizing tissue degeneration and promoting cell preservation.

Specifically, the Lavender essential oil may be provided at approximately 0.3% by volume, the Helichrysum essential oil may be provided at approximately 0.5% by volume, the Cedarwood Atlas essential oil may be provided at approximately 0.2% by volume, and the Frankincense essential oil may be provided at approximately 0.4% by volume.

A method of generating the ointment formulation to treat a skin disorder is also disclosed. According to the method, each of the acts of selecting the ingredient Lanolin, adding the ingredients Lanolin, Zinc Oxide, coconut oil, and petroleum jelly, and adding Lavender, Helichrysum, Cedarwood Atlas, and Frankincense essential oils may be accomplished in an amount toward the upper limit of the respective range for severe levels of skin disorder.

The above features and advantages, and other features and advantages of the present disclosure, will be readily apparent from the following detailed description of the embodiment(s) and best mode(s) for carrying out the described invention when taken in connection with the accompanying drawings and appended claims.

### BRIEF DESCRIPTION OF THE DRAWINGS

FIG. 1 is a flow diagram of a method of formulating the ointment to treat a skin disorder.

### DETAILED DESCRIPTION

The ointment formulation disclosed herein is beneficial for topical treatment of various skin disorders, including various forms of psoriasis and dermatitis, as well as for the general therapeutic treatment of mammal skin. One of the common maladies afflicting skin of mammals is eczema, which is also called atopic dermatitis. Use of the disclosed ointment formulation on a skin of a human being has shown that it was able to relieve itching, dry, and otherwise damaged skin, which are typical conditions associated with the above noted disorders, as well as make the skin generally softer and smoother to the touch.

The disclosed ointment formulation is made up of a number of specific ingredients, each employed in a prescribed proportion, as will be described in detail below. Although the ingredients are mixed together to provide an ointment of substantially homogenous consistency, each of the ingredients imparts to the formulation specific medicinal qualities for treating the subject skin disorders. Additionally, all the concentrations of the ingredients disclosed below permit some variation in order to tailor the ointment formulation to a specific case and severity of the skin disorder.

The ointment formulation includes an occlusive and nourishing ingredient in an amount configured to reduce moisture evaporation from and seal the skin. The specific occlusive and nourishing ingredient in the subject ointment formulation may be Lanolin. Lanolin is additionally selected for its ability to supply the skin with a majority of the 23 proteinogenic, i.e., protein-building, amino acids, which combine into peptide chains to form the building blocks of a vast array of proteins. In an exemplary 126.0 ml volume sample of the ointment formulation, the specific volume of the Lanolin ingredient was 103.53 ml, or 82.17%. Although Lanolin made up 82.17% of the exemplary sample, the allowable range of the Lanolin ingredient in the ointment formulation is approximately 74-85%. The above amount of Lanolin has been found effective to provide the occlusive properties necessary for supporting healing and protection of a person's skin, as well as rapid elimination of associated symptoms of eczema, psoriasis, and dermatitis, such as itching, cracked, burning, and peeling skin.

In persons with more acute skin conditions and disorders, the above concentration of Lanolin was shown to be effective at providing comfort and relief from the itching, cracked, burning, peeling skin, as well as continuous, ongoing healing and protection for current and, new rashes and outbreaks. For less severe conditions, within the range disclosed above, a slightly decreased percentage of Lanolin may be used. The ointment formulation having the specific disclosed volume of Lanolin is for a moderate to severe condition of the skin. Although the amount of Lanolin may be increased for more severe conditions, the disclosed specific concentration of 82.17% is near the maximum that may be used while maintaining the necessary amounts of the other ingredients. Accordingly, as the volume of Lanolin is adjusted for severity of the particular skin condition, the amounts of other ingredients to be described with specificity below would have to be adjusted accordingly. On the other hand, it has been found through experimentation that Lanolin will not provide all the benefits of the subject ointment formulation without inclusion of the other ingredients that are disclosed in detail below.

When the person's skin is prone to itching, burning, peeling, cracking, flacking and splitting it was found that taking measures to optimize retention of moisture is critical for promoting an environment for healing and resultant elimination of such symptoms. A typical alternative approach to treating such skin disorders is to apply a steroid to the skin and then wrap the affected area in plastic wrap and occlusive Coban bandages. It was found, however, that an effect analogous to that of using steroids may be accomplished via the Lanolin ingredient in the disclosed ointment formulation, thus eliminating the controversial use of a steroid in treating a skin disorder. During experimentation with various percentages of Lanolin it was found that too small a volume of the ingredient may cause the ointment to have a decreased ability to retain desirable levels of moisture, particularly when mixed with the other ingredients that are disclosed in detail below.

Lanolin, rich in cholesterol and other skin-friendly sterols, is a refined derivative of the soft, rich, fat-like sebaceous secretion of sheep and is therefore more desirable as a natural moisturizer as opposed to a synthetic substance. Lanolin falls into the lipid-rich emollient class of moisturizers, the oils of which are similar in composition to the oils secreted by human skin. The composition of Lanolin resembles the intercellular lipids of the outermost layer of the skin, the stratum corneum, which consists of cholesterol, cholesterol derivatives, and fatty acids. Normally, water continuously evaporates from the skin's surface such that insufficient rehydration from the lower skin layers leads to a dry, inflexible, and brittle stratum corneum. Such insufficient rehydration may in turn lend itself to symptoms normally associated with dryness and various skin diseases. Lanolin as used in the subject ointment formulation can absorb more than 200% of its weight in water thus effectively rehydrating the skin. Additionally, Lanolin has been shown to cause the water in the skin to build up to its normal level of 10-30% by retarding without completely inhibiting trans-epidermal moisture loss by forming protective films on the skin. Lanolin also has the characteristic of “staying ability”, in which the ingredient's protective layer remains on the skin for a longer duration than other types of moisturizers.

The oils in Lanolin form an emulsion with water that is easily absorbed by the skin and typically prevents excess moisture loss while still allowing the skin to breathe. Since the barrier formed on the skin by Lanolin is not impermeable, air and moisture can still get through, which is as important as the retention of moisture for wound healing and elimination of symptoms of disorders such as eczema, psoriasis, and dermatitis. One of the causes of dry skin is a deficiency of lipids that otherwise fill gaps or splits in the skin, thus permitting water to escape. The use of Lanolin in the ointment formulation according the present disclosure fills in such gaps and facilitates retention of moisture. Furthermore, Lanolin protects and heals the skin via antifungal and antibacterial properties that are inherent to the ingredient, thus preventing infection. As is known, infection is a common condition resulting from cracked, cut, open, and sometimes weeping skin, which is often seen due to continuous scratching of affected itching skin. Moreover, Lanolin is well tolerated by the body, is FDA approved, and undesirable side effects of Lanolin's topical application are minimal, if any.

The ointment formulation includes an anti-pruritic ingredient in an amount configured to reduce itching of the skin, as well as to promote healing of wounds. The specific anti-pruritic ingredient in the subject ointment formulation may be Zinc Oxide, having a chemical formula “ZnO”. In the exemplary 126.0 ml volume sample of the ointment formulation, the specific volume of the Zinc Oxide ingredient was 5.92 ml, or 4.70%. Although the Zinc Oxide made up 4.70% of the exemplary sample, the allowable range of the Zinc Oxide ingredient in the ointment formulation is approximately 4.70-12%. An amount of Zinc Oxide that is lesser than 4.70% has not shown to be effective. However, an increased amount of Zinc Oxide up to the maximum of the above range may be used to enhance the effectiveness of the ointment formulation to sooth the skin and address itching.

Zinc is an antibacterial agent, which plays an important role in healing chronic infection and assisting the body in restoring its ability to heal. Also, the human body has several Zinc dependent enzymes that promote synthesis of collagen, which in turn aids the body in healing skin wounds. Zinc also plays a role in stimulating the transport of vitamin A from the liver to the skin. When Zinc Oxide is applied to the wound area, it provides the body with the extra Zinc that is needed to repair skin cells. Additionally, Zinc Oxide helps also keep the wound area moist and clean, as the Zinc Oxide also possesses antibacterial and astringent properties useful in fending off infection that is commonly associated with skin related disease. Zinc Oxide may also generate a barrier on the skin that prevents progression of a rash, thus reducing inflammation and itching while providing soothing relief. Generally, Zinc deficiency in a human body is known to cause delayed healing of wounds and sores. Moreover, eczema is an inflammatory and chronic disorder of the skin, which is often associated with a deficiency of Zinc.

The ointment formulation includes an antiseptic, i.e., antiviral, antibacterial, and anti-microbial, and anti-oxidant providing, ingredient in an amount configured to protect the skin against infection. The specific antiseptic and anti-oxidant ingredient in the subject ointment formulation may be coconut oil. In the exemplary 126.0 ml volume sample of the ointment formulation, the specific volume of the coconut oil ingredient was 7.40 ml, or 5.87%. Although the coconut oil made up 5.87% of the exemplary sample, the allowable range of the coconut oil ingredient in the ointment formulation is approximately 5.87-9%. The foregoing amount is necessary to provide the anti-microbial, anti-inflammatory, anti-oxidant, itch reducing, tissue repairing and high moisture retaining properties in the ointment formulation. An amount of coconut oil that is lesser than 5.87% has not shown to be effective, while an amount greater than 9% has shown to change the consistency and adversely impact effectiveness of the ointment formulation, mainly by reducing the concentration of other ingredients.

Coconut oil consists of medium chain fatty acids and vitamin E. The acids that are present in coconut oil readily convert themselves into monocaprin and monolaurin, which are anti-microbial and anti-fungal agents. Monocaprin and monolaurin are useful in combating rashes, itching, etc., when acted upon by certain enzymes. Approximately fifty percent of the fatty acids present in coconut oil are in the form of lauric acid, the same as in a mother's milk, thus providing an effective anti-viral, anti-bacterial, and anti-protozoal function. Such anti-viral, anti-bacterial, and anti-protozoal function of coconut oil is highly effective in fighting itchy skin that may result in cuts and scratches, which often lead to infection.

The vitamin E in coconut oil helps to promote healthy skin, repair of skin cells, and facilitates soothing relief and protection of the skin against cracking, flaking and peeling. Coconut oil additionally helps to form a protective barrier on the skin to ward off infection. Coconut oil may soften the skin, relieve dryness, support a natural chemical balance of the skin, and provide natural protection from damaging effects of ultraviolet radiation of the sun. Furthermore, coconut oil does not go rancid, since it is naturally high in antioxidants, which permits the ointment formulation to not require a dedicated preservative. Moreover, coconut oil has been shown to be non-toxic to humans. Employing an organic coconut oil will solidify the above benefits and reduce possibility of unwanted side effects.

The ointment formulation includes a moisturizing ingredient in an amount configured to soothe and reduce dryness of the skin. The specific moisturizing ingredient in the subject ointment formulation may be petroleum jelly. In the exemplary 126.0 ml volume sample of the ointment formulation, the specific volume of the petroleum jelly ingredient was 7.40 ml, or 5.87%. Although the petroleum jelly made up 5.87% of the exemplary sample, the allowable range of the petroleum jelly ingredient in the ointment formulation is approximately 3-7%. The foregoing amount is necessary to provide the moisture retaining properties in the ointment formulation. An amount of petroleum jelly that is lesser than 5.87% has not shown to be effective, while an amount greater than 9% has shown to change the consistency and adversely impact effectiveness of the ointment formulation, mainly by reducing the concentration of other ingredients. In general petroleum jelly hastens healing through a sealing effect the ingredient has on the skin, which inhibits germs from getting into the wound and keeping the injured area supple by preventing the moisture from evaporating from the skin. Petroleum jelly is typically not absorbed into the skin and is also recognized the by the FDA as an over-the-counter skin protectant.

The ointment formulation may also include a plurality of essential oils that may be collectively present in the formulation in a range of up to 2% by volume. Such plurality of essential oils is collectively configured to promote cell preservation and tissue regeneration, provide an antiseptic function, relieve topical discomfort, inflammation, and itching, and impart a physically impacting and pleasing scent. In the subject ointment formulation, each of the plurality of essential oils that will be described in detail below may be up to an approximate maximum of 0.8% of the formulation by volume.

Essential oils generally function in humans according to a dual path. Initially, when an essential oil is applied topically, the oil physically acts upon the body, while simultaneously the scent of the essential oil stimulates the brain. Human beings have the capacity to distinguish 10,000 different scents. Among a multitude of benefits, certain scents can stimulate the brain, prevent drowsiness, reduce anxiety, promote relaxation, initiate healing of the body, and even boost memory. Such scents enter the human body through cilia, i.e., the fine hairs lining the nose, to the limbic system, which is the part of the brain that controls moods, emotions, memory, and learning. Specific effects of different scents can be shown by measuring brain wave frequencies that affect the human body in a multitude of ways. Accordingly, many essential oils impart scents that may be physically impacting, as well as pleasing to the senses of a human being.

An example of a physically impacting scent would be the scent of Lavender increasing alpha waves in the back of the brain, which the human body associates with relaxation. Another example of such a scent would be the fragrance of Jasmine increasing beta waves in the front of the brain, which the human body associates with a more alert state. The human brain responds to a scent in a way similar to how it would respond to a synthetic drug, wherein, dependent upon the stimulation being received, the brain directs specific chemicals to be secreted throughout the body. Such secreted chemicals subsequently result in mood, emotion, memory, and learning alterations, which subsequently result in decreased symptoms of sickness, for example itching, burning, inflammation, etc. Using essential oils for physical well-being may provide tangible advantages over conventional treatments, for example, essential oils typically provide fewer side effects and may also target illnesses more specifically than prescription medication.

The chemical constituents of essential oils have been compared to human blood, in that essential oils are similar with respect to immune and nervous system stimulation, being anti-microbial in nature, containing nutrients and oxygen, and stimulating the regeneration of various tissues. One of the reasons that the essential oils generate such effects is at least in part because of how essential oils are absorbed by the human body. After the essential oil is inhaled, the oil's microscopic droplets are carried to the limbic system of the brain, which is the processing center portion of the brain for reason, emotion, and scent, and to the hypothalamus, which functions as a hormone command center. The essential oil microscopic droplets are also carried to the lungs, where the droplets enter the circulatory system. Some essential oils contain high levels of the chemical constituents sesquiterpenes, which can significantly increase oxygenation and activity in the brain.

The plurality of essential oils in the ointment formulation may include a Lavender essential oil in an amount configured to have an anti-inflammatory and anti-pruritic effect on the skin, as well as reduce itching, and impart the physically impacting (as described in detail above) and an otherwise pleasing scent to the ointment. In the exemplary 126.0 ml volume sample of the ointment formulation, the specific volume of the Lavender essential oil was 0.40 ml, or 0.32%. Although the Lavender essential oil made up 0.32% of the exemplary sample, the allowable range of Lavender essential oil in the ointment formulation is approximately 0.2-0.8%.

Generally, Lavender essential oil calms anxiety and soothes, relaxes, and heals itching skin. The Lavender essential oil is an analgesic, anti-bacterial, anti-inflammatory, anti-microbial, antiseptic and antispasmodic. The preceding characteristics are all invaluable in addressing eczema and other skin conditions involving itching, broken, and irritated skin. The 0.32% concentration of the Lavender essential oil was determined to be the amount that is both safe and effective in calming and healing the skin, combating inflammation, and relieving itching. Additionally, the 0.32% concentration of the Lavender essential oil was shown to lend a mild fragrance to the ointment formulation that is not offensive, overbearing, or irritating which would have an opposite effect from which is being sought. Higher amounts of the Lavender essential oil can be used up to the maximum 0.8% concentration for more severe cases of the subject skin disorders.

The plurality of essential oils in the ointment formulation may also include a Helichrysum essential oil in an amount configured to promote skin regeneration and have a healing effect on the skin. In the exemplary 126.0 ml volume sample of the ointment formulation, the specific volume of the Helichrysum essential oil was 0.6 ml, or 0.48%. Although the Helichrysum essential oil is 0.48% of the exemplary sample, the allowable range of Helichrysum essential oil in the ointment formulation is approximately 0.4-0.8%. The 0.48% concentration of the Helichrysum essential oil was determined to be the amount that is both safe and effective in reducing inflammation, as well as promoting tissue regeneration and new skin cell growth. However, within the permitted range disclosed above, a lesser concentration of the Helichrysum essential oil may be used while still maintaining effectiveness, while a higher concentration can be used for more severe conditions.

Generally, Helichrysum essential oil is a cicatrizant, i.e., promotes tissue regeneration. As a cicatrizant, Helichrysum essential oil aids with the formation of scar tissue and increases the effectiveness of overall wound healing. The Helichrysum essential oil also prevents swelling and discoloration of the skin and, as an emollient, it helps to make the skin smooth, soft, retain moisture, and prevents cracking. Additionally, Helichrysum essential oil is an anti-inflammatory agent, which is highly beneficial for dealing with eczema and other conditions causing skin irritation, because reducing inflammation aids in reducing the irritation. Furthermore, the Helichrysum essential oil is an anti-allergenic, antifungal, and antimicrobial that combats the potential risk of infection that frequently results from the itching and scratching associated with eczema and atopic dermatitis.

The plurality of essential oils in the ointment formulation may additionally include a Cedarwood Atlas essential oil in an amount configured to have a seborrhea reducing and antiseptic effect on the skin. In the exemplary 126.0 ml volume sample of the ointment formulation, the specific volume of the Cedarwood atlas essential oil was 0.25 ml, or 0.20%. Although the Cedarwood Atlas essential oil is 0.48% of the exemplary sample, the allowable range of Cedarwood Atlas essential oil in the ointment formulation is approximately 0.2-0.8%. The 0.20% concentration of the Cedarwood Atlas essential oil was determined to be the amount that is both safe and effective in providing anti-inflammatory, anti-seborrhic (anti-eczema), antiseptic, and stress reducing properties. However, within the permitted range disclosed above, a lesser concentration of the Cedarwood Atlas essential oil may be used while maintaining effectiveness, while a higher concentration can be used for more severe conditions.

Generally, Cedarwood Atlas essential oil is an anti-inflammatory agent that is beneficial when dealing with inflamed and irritated skin that are the typical symptoms of eczema and other skin disorders. Cedarwood Atlas essential oil is also helpful in addressing one of the underlying triggers of eczema and itchy skin, by reducing stress and tension within the human body. Additionally, Cedarwood Atlas essential oil may function as an antiseptic, which is helpful in the healing process with broken and discharging skin.

Furthermore, the plurality of essential oils in the ointment formulation may include a Frankincense essential oil in an amount configured to have a vulnerary effect on the skin by minimizing tissue degeneration and promoting cell preservation. In the exemplary 126.0 ml volume sample of the ointment formulation, the specific volume of the Frankincense essential oil was 0.5 ml, or 0.40%. Although the Frankincense essential oil is 0.40% of the exemplary sample, the allowable range of Frankincense essential oil in the ointment formulation is approximately 0.2-0.8%. The 0.40% concentration of the Frankincense essential oil was determined to be the amount that is both safe and effective in providing the cytophylactic, vulerary, antiseptic, astringent, and anti-anxiety properties. However, within the permitted range disclosed above, a lesser concentration of the frankincense essential oil may be used while still maintaining effectiveness, while a higher concentration may be used for more severe conditions. Lesser concentrations of the Frankincense essential oil have not shown to be effective, while greater concentrations within the permitted range disclosed above can be used for more severe conditions.

Generally, Frankincense essential oil alleviates anxiety and nervous tension brought on by stress, which are common triggers of skin irritation. Frankincense essential oil is also a cytophylactic agent, i.e., serves to encourage growth of new skin cells, and a vulnerary agent, i.e., serves to prevent tissue degeneration and promotes the healing of wounds. Both of the above properties are highly beneficial in addressing the symptoms of eczema and other skin disorders. Frankincense essential oil also functions as an antiseptic, thus acting to reduce infection by preventing or arresting the growth of microorganisms. Additionally, Frankincense essential oil tones and rejuvenates dry skin.

Referring to the drawings, wherein like reference numbers refer to like components, FIG. 1 shows a flow diagram of a method 10 designed to generate the ointment formulation described in detail above. As described, the ointment formulation is intended to treat a skin disorder and provide relief of topical discomfort and symptoms of eczema on a skin of a mammal.

The method commences in frame 20, where it includes selecting the occlusive and nourishing ingredient Lanolin in an amount configured to reduce moisture evaporation from and seal the skin. As described above, adding the ingredient Lanolin may be accomplished at approximately 82% of the ointment by volume. Following frame 20, the frame advances to frame 30. In frame 30, the method includes adding the anti-pruritic ingredient Zinc Oxide in an amount configured to reduce itching. As described above, adding the ingredient Zinc Oxide may be accomplished at approximately 5% of the ointment by volume.

After frame 30 the method proceeds to frame 40, where it includes adding the antiseptic and anti-oxidant ingredient coconut oil in an amount configured to protect the skin against infection. As described above, adding the ingredient coconut oil may be accomplished at approximately 6% of the ointment by volume. Following frame 40, the method advances to frame 50 where it includes adding the moisturizing ingredient petroleum jelly in an amount configured to soothe, i.e., reduce dryness of, the skin. As described above, adding the ingredient petroleum jelly may be accomplished at approximately 6% of the ointment by volume. The method concludes in frame 70 where it includes mixing thoroughly the ingredients Lanolin, Zinc Oxide, coconut oil, and petroleum jelly to generate the ointment formulation.

Prior to concluding in frame 70, the method may include adding the plurality of essential oils collectively in the range of up to 2% of the ointment formulation by volume in frame 60. Accordingly, in frame 60 the method may include adding the Lavender essential oil in an amount configured to have an anti-inflammatory and anti-pruritic effect on the skin, reduce itching, and impart the physically impacting and pleasing scent to the ointment formulation. Also in frame 60, the method may include adding the Helichrysum essential oil in an amount configured to promote skin regeneration and have a healing effect on the skin. Additionally, in frame 60 the method may include adding the Cedarwood Atlas essential oil in an amount configured to have a seborrhea reducing and antiseptic effect on the skin.

Furthermore, in frame 60 the method may include adding the Frankincense essential oil in an amount configured to have a vulnerary effect on the skin by minimizing tissue degeneration and promoting cell preservation. Following frame 60, the method may proceed to frame 70 where it includes mixing thoroughly the plurality of essential oils with the ingredients Lanolin, Zinc Oxide, coconut oil, and petroleum jelly to generate the ointment formulation. According to the method 10, each of the acts of selecting the ingredient Lanolin, adding the ingredients Lanolin, Zinc Oxide, coconut oil, and petroleum jelly, and adding Lavender, Helichrysum, Cedarwood Atlas, and Frankincense essential oils may be accomplished in an amount toward the upper limit of the respective range for severe levels of skin disorder.

The detailed description and the drawings or figures are supportive and descriptive of the invention, but the scope of the invention is defined solely by the claims. While some of the best modes and other embodiments for carrying out the claimed invention have been described in detail, various alternative designs and embodiments exist for practicing the invention defined in the appended claims.

1. An ointment formulation for treating a skin of a mammal, the ointment formulation comprising:
an occlusive and nourishing ingredient in an amount configured to reduce moisture evaporation from and seal the skin;
an anti-pruritic ingredient in an amount configured to reduce itching of the skin;
an antiseptic and anti-oxidant providing ingredient in an amount configured to protect the skin against infection; and
a moisturizing ingredient in an amount configured to soothe and reduce dryness of the skin.

2. The ointment formulation according to claim 1, wherein:
the occlusive and nourishing ingredient is Lanolin;
the anti-pruritic ingredient is Zinc Oxide;
the antiseptic and anti-oxidant providing ingredient is coconut oil; and
the moisturizing ingredient is petroleum jelly.

3. The ointment formulation according to claim 2, wherein:
Lanolin is in the range of 74-85% by volume;
Zinc Oxide is in the range of 4-12% by volume;
coconut oil is in the range of 5-9% by volume; and
petroleum jelly is in the range of 3-7% by volume.

4. The ointment formulation according to claim 3, wherein:
Lanolin is approximately 82% by volume;
Zinc Oxide is approximately 5% by volume;
coconut oil is approximately 6% by volume; and
petroleum jelly is approximately 6% by volume.

5. The ointment formulation according to claim 1, further comprising a plurality of essential oils collectively in a range of up to 2% by volume.

6. The ointment formulation according to claim 5, wherein the plurality of essential oils is collectively configured to promote cell preservation and tissue regeneration, provide an antiseptic function, relieve topical discomfort, inflammation, and itching, and impart a physically impacting and pleasing scent.

7. The ointment formulation according to claim 6, wherein each of the plurality of essential oils is in a range up to an approximate maximum of 0.8% of the formulation by volume.

8. The ointment formulation according to claim 7, wherein the plurality of essential oils includes:
a Lavender essential oil in an amount configured to have an anti-inflammatory and anti-pruritic effect on the skin, reduce itching, and impart the physically impacting and pleasing scent to the ointment formulation;
a Helichrysum essential oil in an amount configured to promote skin regeneration and have a healing effect on the skin;
a Cedarwood Atlas essential oil in an amount configured to have a seborrhea reducing and antiseptic effect on the skin; and
a Frankincense essential oil in an amount configured to have a vulnerary effect on the skin by minimizing tissue degeneration and promoting cell preservation.

9. The ointment formulation according to claim 8, wherein:
the Lavender essential oil is approximately 0.3% by volume;
the Helichrysum essential oil is approximately 0.5% by volume;
the Cedarwood Atlas essential oil is approximately 0.2% by volume; and
the Frankincense essential oil is approximately 0.4% by volume.

10. The ointment formulation according to claim 1, wherein the coconut oil is an organic substance.

11. A method of generating an ointment formulation for treating a skin of a mammal, the method comprising:
selecting an occlusive and nourishing ingredient Lanolin in an amount configured to reduce moisture evaporation from and seal the skin;
adding an anti-pruritic ingredient Zinc Oxide in an amount configured to reduce itching;
adding an antiseptic and anti-oxidant ingredient coconut oil in an amount configured to protect the skin against infection;
adding a moisturizing ingredient petroleum jelly in an amount configured to soothe and reduce dryness of the skin; and
mixing thoroughly the ingredients Lanolin, Zinc Oxide, coconut oil, and petroleum jelly to generate the ointment formulation.

12. The method according to claim 11, wherein:
said selecting the ingredient Lanolin is accomplished in the range of 74-85% of the ointment by volume;
said adding the ingredient Zinc Oxide is accomplished in the range of 4-12% of the ointment by volume;
said adding the ingredient coconut oil is accomplished in the range of 5-9% of the ointment by volume; and
said adding the ingredient petroleum jelly is accomplished in the range of 3-7% of the ointment by volume.

13. The method according to claim 12, wherein:
said adding the ingredient Lanolin is accomplished at approximately 82% of the ointment by volume;
said adding the ingredient Zinc Oxide is accomplished at approximately 5% of the ointment by volume;
said adding the ingredient coconut oil is accomplished at approximately 6% of the ointment by volume; and
said adding the ingredient petroleum jelly is accomplished at approximately 6% of the ointment by volume.

14. The method according to claim 12, further comprising adding a plurality of essential oils collectively in a range of up to 2% of the ointment by volume and mixing thoroughly the plurality of essential oils with the ingredients Lanolin, Zinc Oxide, coconut oil, and petroleum jelly to generate the formulation.

15. The method according to claim 14, wherein the plurality of essential oils is collectively configured to promote cell preservation and tissue regeneration, provide an antiseptic function, relieve topical discomfort, inflammation, and itching, and impart a physically impacting and pleasing scent.

16. The method according to claim 15, wherein each of the plurality of essential oils is in a range up to an approximate maximum of 0.8% of the formulation by volume.

17. The method according to claim 16, wherein the plurality of essential oils includes:
a Lavender essential oil in an amount configured to have an anti-inflammatory and anti-pruritic effect on the skin, reduce itching, and impart the physically impacting and pleasing scent to the ointment formulation;
a Helichrysum essential oil in an amount configured to promote skin regeneration and have a healing effect on the skin;
a Cedarwood Atlas essential oil in an amount configured to have a seborrhea reducing and antiseptic effect on the skin; and
a Frankincense essential oil in an amount configured to have a vulnerary effect on the skin by minimizing tissue degeneration and promoting cell preservation.

18. The method according to claim 17, wherein:
said adding the Lavender essential oil is accomplished at approximately 0.3% of the ointment by volume;
said adding the Helichrysum essential oil is accomplished at approximately 0.5% of the ointment by volume;
said adding the Cedarwood Atlas essential oil is accomplished at approximately 0.2% of the ointment by volume; and
said adding the Frankincense essential oil is accomplished at approximately 0.4% of the ointment by volume.

19. The method according to claim 17, wherein each of said selecting the ingredient Lanolin, said adding the ingredients Lanolin, Zinc Oxide, coconut oil, and petroleum jelly, and said adding Lavender, Helichrysum, Cedarwood Atlas, and Frankincense essential oils is accomplished in an amount toward the upper limit of the respective range for severe levels of skin disorder.

20. The method according to claim 11, wherein the coconut oil is an organic substance.
